SEQUENCE LISTING (14 sequences)

SEQ ID NO: 1 - DNA, 21 bp, Artificial Sequence
  Primer
cgcctgagcaaagtaaatgag

SEQ ID NO: 2 - DNA, 19 bp, Artificial Sequence
  Primer
gccctcgatatagcggatg

SEQ ID NO: 3 - DNA, 25 bp, Artificial Sequence
  Primer
gaataaagatggggttctcatatcc

SEQ ID NO: 4 - DNA, 21 bp, Artificial Sequence
  Primer
aacttcagcaaactcaaactt

SEQ ID NO: 5 - DNA, 25 bp, Artificial Sequence
  Primer
cggcaactctagtatttaggataac

SEQ ID NO: 6 - DNA, 24 bp, Artificial Sequence
  Primer
caaataaaatgacaaggcacgatt

SEQ ID NO: 7 - DNA, 20 bp, Artificial Sequence
  Primer
ttctccccttttcccactga

SEQ ID NO: 8 - DNA, 25 bp, Artificial Sequence
  Primer
caaacgcaatcactatctataccat

SEQ ID NO: 9 - DNA, 19 bp, Artificial Sequence
  Primer
ggaccagtacccgcacttg

SEQ ID NO: 10 - DNA, 20 bp, Artificial Sequence
  Primer
aatccgggtggtccttcttg

SEQ ID NO: 11 - DNA, 23 bp, Artificial Sequence
  Primer
cactaaggaaggaattgggaaca

SEQ ID NO: 12 - DNA, 25 bp, Artificial Sequence
  Primer
gggattaaaatcaagagcatcattg

SEQ ID NO: 13 - DNA, 18 bp, Artificial Sequence
  Primer
cgtttcgtgtccctttca

SEQ ID NO: 14 - DNA, 25 bp, Artificial Sequence
  Primer
cctcttgttcattcttgttcgtatt

(19) United States
(12) Patent Application Publication
(10) Pub. No.: US 2021/0322635 A1
(43) Pub. Date: Oct. 21, 2021

(54) TISSUE SCAFFOLD

(71) Applicant: Raft Enterprises Ltd., Northwood, Middlesex (GB)

(72) Inventors: Prasad Sawadkar, Northwood, Middlesex (GB); Elena García-Gareta, Northwood, Middlesex (GB)

(21) Appl. No.: 17/267,910

(22) PCT Filed: Aug. 16, 2019
(86) PCT No.: PCT/GB2019/052313

§ 371 Date: Feb. 11, 2021

(30) Foreign Application Priority Data
Aug. 17, 2018 (GB) 1813461.9

(51) Int. Cl. A61L 27/26; A61L 27/38; C12N 5/0775

(52) U.S. Cl. CPC A61L 27/26; A61L 27/3834; C12N 2533/50; C12N 2533/30; C12N 5/0667

(57) ABSTRACT

A tissue scaffold is described. The tissue scaffold comprises a synthetic polymer and one or more natural polymers selected from elastin, fibrin and collagen. The synthetic polymer may be in the form of a fibrous matrix coated with the at least one natural polymer.

This invention concerns tissue scaffolds having synthetic and natural polymers, methods for forming such tissue scaffolds and applications of such tissue scaffolds, such as in tissue engineering and tissue regeneration.

In contemporary reconstructive surgery, a paradigm shift is taking place to overcome the limitations of alloplastic tissue repair and tissue grafting by using a tissue engineering approach (1). The concept of tissue engineering was initiated three decades ago and uses a combination of scaffolds fabricated from biomateriais and cells/stem cells for clinical therapies.

A scaffold acts as an initial structural matrix for cells to achieve tissue regeneration by neo tissue formation and remodelling. Its architecture and composition influences cell behaviour, therefore encouraging the differentiation of cells into functional tissue (2, 3). Polymeric scaffolds are either fabricated from natural or synthetic polymeric sources and both approaches have their advantages and disadvantages. Natural polymers are biologically and immunologically recognised with self-degradable moieties, but they can be mechanically weak structures (4). In contrast, synthetic polymers have weaker biological and stronger mechanical properties, but their mechanical properties are altered upon degradation by hydrolysis (5, 6). No polymer by itself is ideal for tissue regeneration purposes.

Polycaprolactone (PCL) is an aliphatic oligoesteric polymer as it is fabricated by ring opening polymerisation process of ε-caprolactone in the presence of a catalyst (7). It is biocompatible, biodegradable, and FDA approved. PCL also exhibits various advantages in comparison to other aliphatic polymers such as polylactic acid (PLA) and polyglycolic acid (PGA): 1) PCL does not have isomeric forms like PLA, e.g. poly-L-lactic acid (PLLA), poly-D-lactide acid (PDLA), poly-DL-lactic acid (PDLLA); 2) its melting point is lower than those of PLA and PGA which gives PCL a wide range of mechanical and viscoelastic properties to mimic native tissue (8). However, it has the disadvantage of hydrophobicity which causes poor wettability and cell attachment. Moreover, it has a slow degradation rate. (8, 9).

In order to implant functional tissue constructs in vivo or produce native mimicking tissue in vitro, scaffolds can be populated with relevant cells to speed up tissue remodelling via cell signalling mechanisms. Hence, cell seeded scaffolds act as a template for neo-tissue formation. (10, 11) It is well established that stem cells and their multipotent properties have the ability to repair damaged tissue and restore its function. Stem cells can be derived from bone marrow, peripheral blood, amniotic fluid, umbilical cord, embryonic and adipose sources. Also, somatic cells can be manipulated to form stem cells as an induced pluripotent stem cell (iPSC). Mesenchymal stem cells (MSC) from adipose origin are advantageous as they are harvested by minimally invasive methods with low morbidity and maximum yield (12). In addition, MSC can maintain their multipotency by self-renewing themselves, but upon external chemical and/or mechanical stimuli they can be differentiated into various cell linages such as adipogenic, osteogenic, chondrogenic, myogenic, etc. (13).

The inventors have appreciated how disadvantages associated with the use of synthetic polymers such as PCL can be reduced or eliminated and how scaffolds based on synthetic polymers may be modified to enhance their physical and biological properties to facilitate various tissue regeneration applications.

According to the invention, there may be provided a tissue scaffold comprising a synthetic polymer (or artificial polymer) and one or more natural polymers (or biopolymers), and methods for making such scaffolds. Optional additional features of such tissue scaffolds and optional additional method steps for forming such tissue scaffolds are described below.

The synthetic polymer may be polycaprolactone. However, other synthetic polymers suitable for use in tissue engineering may be applicable, such as polyesters, polyanhydrides, polyphosphazenes, polyurethane, and poly (glycerol sebacate). Suitable polyesters include polylactide (PLA), polyglycolide (PGA), and their copolymer poly(lactide-co-glycolide) (PLGA), poly(lactide-co-caprolactone) and polyacrylonitrile (PAN).

The at least one natural polymer may comprise one or more natural polymers selected from elastin, collagen and fibrin. The inventors have appreciated that using one of these natural polymers, two of the natural polymers or all three of these natural polymers can result in different properties that may be beneficial for different applications.

The scaffolds may comprise a synthetic polymer matrix and:
i) fibrin (F);
ii) elastin (E);
iii) collagen (C);
iv) fibrin and elastin (FE);
v) fibrin and collagen (FC);
vi) elastin and collagen (EC); or
vii) elastin and collagen and fibrin (ECF).

The natural polymers may be extracted or derived from a natural source. For example, the natural polymers may be derived from a mammalian source. Alternatively, the natural polymer may be recombinant.

Elastin may be obtained from a bovine source, such as bovine neck ligament, or a human source. Collagen may be obtained from a rodent source, such as rat tail.

The natural polymers may be treated before contacting with the synthetic polymers. For example, the polymers may be solubilised or enzymatically treated to make them more amenable for the methods described herein. In the case of fibrin, this may be formed by contacting fibrinogen with thrombin. More detail about how the polymers may be treated or formed, is provided below.

According to the invention, there may be provided a tissue scaffold comprising a synthetic polymer and natural polymers selected from fibrin and: i) collagen; and/or ii) elastin.

According to the invention, there may be provided a method for forming a tissue scaffold comprising contacting a synthetic polymer with natural polymers selected from fibrin and: i) collagen; and/or ii) elastin.

The synthetic polymer may be in the form of a matrix. The matrix may be fibrous, for example it may be in the form of a fibrous mat. The fibres may be randomly oriented. The fibers may be aligned. The scaffold may comprise nanofibers (e.g. the matrix may comprise nanofibers), with a diameter less than or equal to 1000 nanometres (nm). However, the scaffold may comprise microfibers (e.g. the matrix may comprise microfibers). The matrix may not be in the form of a foam.

The fibres may have a maximum diameter less than or equal to 1 millimetre, less than or equal to 500 micrometres (μm), less than or equal to 50 μm or less than or equal to 10 μm, less than or equal to 1 μm, less than or equal to 750 nm, less than or equal to 500 nm, less than or equal to 250 nm or less than or equal to 100 nm.

The fibres may have a mean diameter less than or equal to 1 millimetre, less than or equal to 500 micrometres (μm), less than or equal to 50 μm or less than or equal to 10 μm, less than or equal to 1 μm, less than or equal to 750 nm, less than or equal to 500 nm, less than or equal to 250 nm or less than or equal to 100 nm.

The fibres may have a mean or maximum diameter of 10 nm to 500 μm. The fibres may have a mean or maximum diameter of 100 nm to 100 μm. The fibres may have a mean or maximum diameter from 1 μm to 250 μm. The fibres may have a mean or maximum diameter of 250 nm to 10 μm.

The matrix may be formed by electrospinning. Advantageously, electrospinning may result in fibre diameters that are in the size range of the extracellular matrix microstructures. Scaffolds may be formed by electrospinning a solution or melt comprising the synthetic polymer. This may occur prior to contact with the one or more natural polymers. Alternatively, scaffolds may be formed by electrospinning a solution, suspension or melt comprising the synthetic polymer and a natural polymer (or a natural polymer precursor, such as fibrinogen).

Although scaffolds could be formed using other techniques such as particulate leaching, and thermally induced phase separation, or by solid freeform fabrication techniques such as 3D-printing stereolithography, selective laser sintering fused deposition modelling/3D plotting or direct writing, it is contemplated that the scaffold may be fabricated by not utilising one or more of these techniques.

Scaffolds may comprise the matrix which is coated with the one or more natural polymers. For example, the tissue scaffold may be formed by immersing the synthetic polymer matrix in a solution or suspension comprising the one or more natural polymer. This may result in a scaffold comprising a matrix which is coated, preferably substantially evenly, with the one or more natural polymers. Other suitable methods may include spraying the matrix with a solution or suspension comprising the elastin, collagen and/or fibrin. The pre-formed or pre-fabricated matrix may be contacted by the one or more natural polymers. The coating of the one or more natural polymers onto the synthetic polymer matrix may be referred to as post-coating. The scaffold may be formed without any chemical cross-linking to, for example, cross-link the synthetic polymer to the one or more natural polymer.

In addition to the apparent beneficial physical properties described herein, post-coating of a synthetic polymer matrix may provide a more flexible, convenient and less expensive method to form a tissue scaffold. For example, it may enable suitable scaffolds to be manufactured using a pre-existing, off-the-shelf, synthetic polymer matrix.

According to the invention, there may be provided a tissue scaffold comprising a fibrous synthetic polymer matrix coated with at least one natural polymer selected from elastin, fibrin and collagen.

According to the invention, there may be provided a method for forming a tissue scaffold, comprising coating a fibrous synthetic polymer matrix with one or more natural polymers selected from elastin, fibrin and collagen.

As an alternative to post-coating, the scaffolds may be manufactured by forming a matrix that comprises the synthetic polymer and the at least one natural polymer. For example, the scaffolds may be manufactured by forming fibres comprising the synthetic polymer and the at least one natural polymer. This may be achieved, for example, by forming fibres from a solution or suspension comprising both the synthetic polymer and the at least one natural polymer. The fibres may be formed by electrospinning, or other techniques such as gas-blowing or gas-brushing.

A solution or suspension of fibrin may be formed by contacting fibrinogen with thrombin. For example, a fibrinogen solution (for example a 2% fibrinogen solution) may be dissolved in PBS with thrombin. Formation of fibrin may thus occur before contact with the matrix. However, it is appreciated that a tissue scaffold of the invention may be formed by contacting the matrix with a solution or suspension comprising fibrinogen. Formation of fibrin may then occur in situ, on the matrix, by addition of thrombin. The fibrin may not be in the form of a hydrogel.

A solution or suspension of collagen may be formed by mixing collagen, such as rat tail collagen, with 10× minimal essential medium and neutralising by adding sodium hydroxide.

A solution or suspension of elastin may be formed by solubilising elastin. Solubilised elastin, or soluble elastin, may also be referred to as hydrolysed elastin or elastin peptides.

Common methods of solubilising elastin include treating it with 0.25 M oxalic acid at 100° C., or treating it with 1M KOH in 80% ethanol. In addition, proteolytic enzymes capable of degrading elastic fibres, including serine-type elastases from polymorphonuclear leukocytes and several metallo-elastases of monocyte/macrophage origin, also result in solubilised elastin. Examples of hydrolysed forms of elastin are show in the table below.

| Type | Preparation Method | Molecular mass |
|---|---|---|
| α | Oxalic acid solubilisation | Heterogeneous mixture, average 60 kDa |
| β | Oxalic acid solubilisation | Heterogeneous mixture, average 3-10 |
| | | kDa |
| κ | KOH solubilisation | Heterogeneous mixture, average 70 kDa |
| PSP | Pepsin solubilisation | Heterogeneous mixture, average 25 KDa |
| ASP | Acid solubilisation | Heterogeneous mixture, average 25 kDa. |
| ESP | Elastase solubilisation | Heterogeneous mixture |

Elastin peptides obtained after oxalic acid hydrolysis can be coacervated after suspension in 10 mM sodium acetate with 10 mM NaCl set to pH 5.5 with acetic acid, followed by heating and centrifugation at 37° C. As a result of this, two fractions are formed, α-elastin (a viscous coacervate) and β-elastin (in the supernatant).

Although conventional tissue engineering applications have focussed on using soluble, fractionated elastin, the inventors have surprisingly found that a suitable elastin composition can be formed by simply exposing the elastin to a solubilising agent and using the product of that step, without a requirement to separate or isolate fractions of elastin, such as separating or isolating α-elastin and β-elastin. The resulting elastin may thus be considered crude or unfractionated. Advantageously, the invention may avoid the time, inconvenience and expense associated with isolating the α-elastin fraction. The invention may also improve total yield, as a step to separate α-elastin and β-elastin can be avoided.

Consequently, the elastin that is contacted with the synthetic polymer matrix may be unfractionated and comprises soluble elastin. Optionally, the elastin may also comprise insoluble elastin The unfractionated elastin may thus be crude elastin in which there has been no purification, isolation, separation or refinement of one or more elastin fractions, or one or more different forms of elastin which result from the contact of elastin with a solubilising agent. For instance, there may not have been isolation of one or more soluble elastin fractions, or separation of an insoluble elastin fraction from a soluble elastin fraction. Consequently, the composition may comprise different soluble forms of elastin. The composition may comprise elastin that has not been solubilised. The composition may thus comprise both soluble and insoluble forms of elastin. For example, following contact with the solubilising agent, the elastin may not be subjected to centrifugation. There may be no fractionation, purification, isolation, separation or refinement of the elastin following contact with the solubilising agent.

The elastin may be, or may have been, solubilised by contacting with acid, most preferably oxalic acid. The elastin may be solubilised at a temperature less than 100° C., preferably at a temperature of less than or equal to 50° C., more preferably at a temperature of 15 to 30° C., such as room temperature.

The acid, preferably oxalic acid, may be at less than 1M, preferably less than 0.75M, more preferably at 0.5M, or less than 0.5M. The acid may be at least 0.25M. For example, the acid may be at 0.2M to 1M, for example 0.25M to 0.75M.

Solubilisation of the elastin, or contact of the elastin with acid (preferably oxalic acid), may take place for at least 30 seconds, more preferably at least one minute. For example, the solubilisation or contact with acid may take place for about 1 to 3 minutes. The solubilisation, or contact with acid, may take place for up to 5 minutes. This contrasts with the conventional treatment of elastin with oxalic acid which typically takes place for about 1 hour.

According to the invention, there may be provided a tissue scaffold comprising a synthetic polymer and at least one natural polymer comprising elastin, and wherein the elastin is unfractionated and comprises soluble elastin.

According to the invention, there may be provided a method for forming a tissue scaffold comprising contacting a synthetic polymer with at least one natural polymer comprising elastin, and wherein the elastin is unfractionated and comprises soluble elastin.

Scaffolds of the invention are preferably not hydrogels or do not comprise hydrogels. The scaffolds may be dry and thus substantially free of water. There may be 5% or less (by weight) of water, 3% or less (by weight) of water, 2% or less (by weight) of water, or 1% or less (by weight) of water.

The scaffold may be lyophilised, or dried by any other suitable means. Lyophilisation may occur for at least 24 hours, such as at least 48 hours.

The matrix may be, or may have been, contacted with the one or more natural polymers, in the absence of any cells. Once the matrix has been contacted with the one or more natural polymers, the resulting scaffold may be seeded with one or more cells. Seeding of the scaffold may occur in vitro or ex viva Consequently the invention may provide a scaffold of the invention in combination with one or more cells. Seeding the scaffold with cells prior to application to a subject may assist in speeding up tissue remodelling. The cells may be stem cells. Suitable stem cells include human adipose-derived stem cells (hADSCs).

According to the invention, there may be provided a tissue or cell culture comprising a tissue scaffold as described herein.

The scaffolds may have a mean pore diameter of 120 μm or less. Scaffolds may have a mean pore diameter of 100 μm or less. Scaffolds may have a mean pore diameter of 10 μm or greater. Scaffolds may have a mean pore diameter of 20 μm or greater. For instance, scaffolds may have a mean pore diameter of 10 to 150 μm. Scaffolds may have a mean pore diameter of 10 to 100 μm. Scaffolds may have a mean pore diameter of 1 to 75 μm, such as 1 to 50 μm.

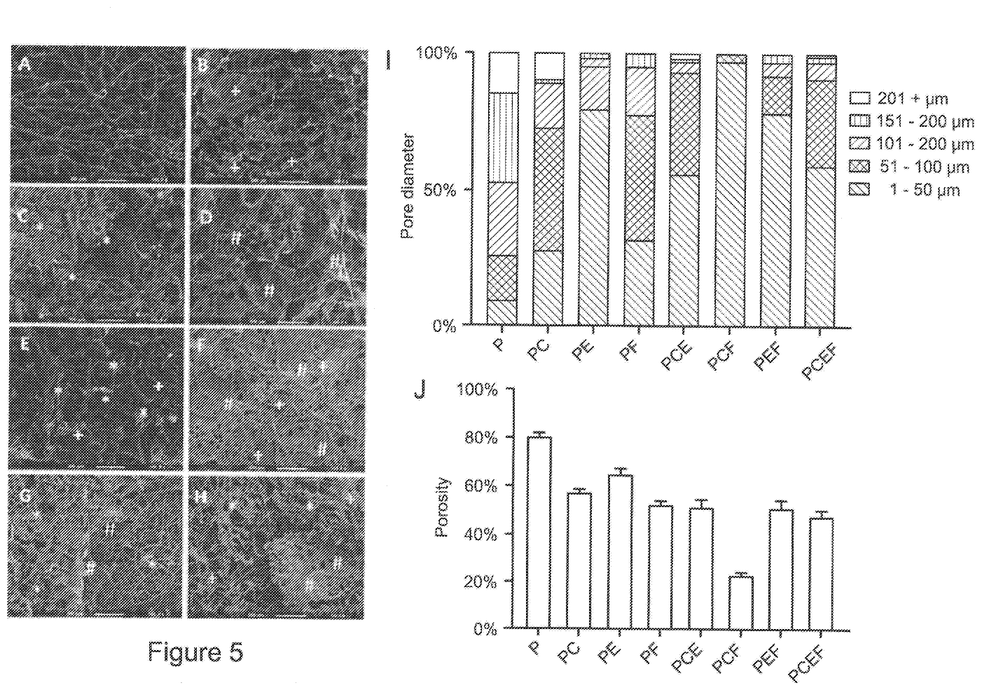

Advantageously, the pore size distribution may be adjusted by inclusion of different combinations of natural polymers. This is demonstrated, for instance, in FIG. 5. Some scaffolds, such as those comprising: elastin; collagen & elastin; collagen & fibrin; elastin & fibrin; and collagen, elastin & fibrin may have at least 50% of their pores with a diameter of 1 to 50 μm. Some scaffolds, such as those comprising: elastin; collage & fibrin; elastin & fibrin, may have at least 75% of their pores with a diameter of 1 to 50 μm.

The scaffolds may have at least 50% of their pores with a diameter of 1 to 100 μm.

Characteristics of scaffolds such as pore size and porosity may be calculated using appropriate readily-available software. For example, ND (“Nearest Distance”) is an ImageJ plugin that was developed to calculate the average size and distance between pores and their nearest neighbours in porous scaffolds. DiameterJ is another example of an ImageJ plugin that can be used to measure pore parameters. Microscopic images of the scaffold (e.g. SEM images) may be used as input.

The porosity of scaffolds may be at least 20%. The porosity of the scaffolds may be at least 40%. The porosity of the scaffolds may be less than 80%. The porosity may vary depending on the specific combination of natural polymers used. This is demonstrated, for instance in FIG. 5.

The scaffolds may be sterile. The scaffolds may be packaged, for example in sealed packaging.

Scaffolds of the invention may not be multi-layer scaffolds, such as scaffolds formed from alternating layers of synthetic polymer and at least one natural polymer. For example, the scaffold may comprise a single layer of a synthetic polymer matrix, which is coated with the at least one natural polymer.

The tissue scaffold may be for use as a medicament. The tissue scaffold may be administered or applied to a human subject.

The tissue scaffold may be for use in tissue engineering. The tissue scaffold may be for use in promoting tissue healing, regeneration or repair.

According to the invention, there may be provided a method for promoting tissue regeneration, tissue healing or tissue repair comprising applying a tissue scaffold as described herein, to a patient in need thereof.

The scaffold may be for use in therapeutic applications. The scaffold may be used in none therapeutic applications or methods, such as cosmetic applications or methods. For instance, the scaffold may be used for improving the appearance of a tissue or organ, such as skin.

According to the invention, there may be provided use of a tissue scaffold as described herein, in the manufacture of a medicament for promoting tissue regeneration, tissue healing or tissue repair.

Some scaffolds may be more suitable for certain types of tissue regeneration than others. For instance, scaffolds could be tailored to the regenerative ability of the native tissues.

Scaffolds which are less resistant to degradation may be more applicable to large organ engineering, such as skin and liver, which tend to have strong regeneration and repair abilities. Scaffolds comprising elastin and elastin and fibrin, for example, may be suitable.

Scaffolds which are more resistant to degradation may be more applicable to soft connecting tissue engineering, such as tendon and ligaments, which tend to have weaker regeneration properties. For example, scaffolds comprising: collagen; fibrinogen; collagen & elastin; collagen, elastin & fibrin may be suitable.

Some scaffolds may be more pro-angiogenic than others. For example, scaffolds comprising fibrin or fibrin & collagen may be particularly pro-angiogenic. Scaffolds comprising elastin, or elastin and collagen, may be moderately pro-angiogenic, whereas scaffolds comprising elastin and fibrin may not be particularly pro-angiogenic. The ability to promote angiogenesis may be more crucial for some tissue engineering applications than others.

The mechanical properties of the scaffolds may also have a bearing on their potential applications. For example, stronger scaffolds may have the potential for orthopaedic tissue engineering. Stronger scaffolds may comprise collagen and fibrin. Weaker scaffolds may be applicable for soft tissue engineering, which may include scaffolds comprising: elastin; collagen & elastin; elastin & fibrin; collagen, elastin & fibrin.

Examples, which may be according to the invention, are listed in the following numbered clauses.

1. A tissue scaffold comprising a synthetic polymer and one or more natural polymers selected from elastin, fibrin and collagen.

2. A tissue scaffold according to clause 1, wherein the synthetic polymer is in the form of a matrix.

3. A tissue scaffold according to clause 1 or clause 2, comprising fibers, optionally wherein the synthetic polymer is in the form of a fibrous matrix.

4. A tissue scaffold according to clause 3, wherein the fibres have a mean diameter of 10 nm to 500 μm, 100 nm to 100 μm, 1 μm to 250 μm or 250 nm to 10 μm.

5. A tissue scaffold according to clause 3 or clause 4, wherein the fibres have a maximum diameter of 10 nm to 500 μm, 100 nm to 100 μm, 1 μm to 250 μm or 250 nm to 10 μm.

6. A tissue scaffold according to any preceding clause, comprising nanofibers, optionally wherein the synthetic polymer is in the form of a nanofibrous matrix.

7. A tissue scaffold according to any preceding clause, comprising electrospun fibres, optionally wherein the synthetic polymer is in the form of an electrospun matrix.

8. A tissue scaffold according to any of clauses 2 to 5, in which the matrix is coated with the one or more natural polymers.

9. A tissue scaffold according to any preceding clause which is lyophilised.

10. A tissue scaffold according to any preceding clause, comprising elastin,

11. A tissue scaffold according to clause 10, wherein the elastin is unfractionated and comprises soluble elastin.

12, A tissue scaffold according to any preceding clause, comprising fibrin. β. A tissue scaffold according to any preceding clause comprising collagen.

14. A tissue scaffold according to any preceding clause, wherein the synthetic polymer comprises PCL.

15. A tissue scaffold according to any preceding clause, which is ex vivo or in vitro.

16. A tissue scaffold according to any preceding clause, in combination with at least one cell, preferably wherein the scaffold has been seeded with the at least one cell.

17. A tissue scaffold according to clause 16, wherein the at least once cell comprises a stem cell, such as hADSC.

18. A tissue scaffold according to any preceding clause, which has a mean pore diameter of 1 to 150 μm, or 10 to 100 μm.

19. A tissue scaffold according to any preceding clause, which has at least 50% of pores with a diameter of 1 to 100 μm.

20. A tissue scaffold according to any preceding clause, with a porosity of at least 20% or at least 40%.

21. A method for forming a tissue scaffold comprising contacting a synthetic polymer with one or more natural polymers selected from elastin, fibrin and collagen.

22. A method according to clause 21, comprising forming a synthetic polymer matrix, or wherein the tissue scaffold is in the form of a synthetic polymer matrix.

23. A method according to clause 22 comprising coating the matrix with the one or more of the natural polymers.

24. A method according to clause 22 or clause 23, comprising contacting the matrix with a solution or suspension comprising the one or more of the natural polymers.

25. A method according to clause 24, comprising immersing the synthetic polymer matrix in the solution or suspension comprising the one or more of the natural polymers.

26. A method according to any of clauses 22 to 25, wherein the polymer matrix is fibrous.

27. A method according to clause 26, wherein the matrix comprises nanofibers.

28. A method according to clause 26 or 27, wherein the polymer matrix has been formed by electrospinning, or wherein the method comprises electrospinning a solution comprising the synthetic polymer.

29. A method according to any of clauses 21 to 28, comprising lyophilisation following contact of the synthetic polymer with the one or more natural polymers.

30. A method according to any of clauses 21 to 29, wherein the synthetic polymer comprises polycaprolactone (PCL).

31. A method according to any of clauses 21 to 30, wherein the one or more natural polymers comprises elastin.

32. A method according to clause 31, wherein the elastin is unfractionated and comprises soluble elastin.

33. A method according to clause 32, comprising a step of solubilising elastin.

34. A method according to clause 32 or clause 33, wherein the elastin is, or has been, solubilised by contacting with oxalic acid.

35. A method according to any of clauses 32 to 34, wherein the elastin is, or has been, solubilised at a temperature less than 100° C.

36. A method according to any of clauses 32 to 35, wherein the step of solubilising the elastin is, or has been, carried out at a temperature less than or equal to 50° C.,

37. A method according to any of clauses 32 to 36, wherein the step of solubilising the elastin is, or has been, carried out at a temperature of 15 to 30° C.

38. A method according to any of clauses 32 to 37, wherein the elastin is, or has been, solubilised for a period of 30 seconds to 5 minutes, optionally 1 to 3 minutes.

39. A method according to any of clauses 31 to 38, wherein the elastin comprises insoluble elastin.

40. A method according to any of clauses 21 to 39, wherein the one or more natural polymers comprises fibrin.

41. A method according to any of clauses 21 to 40, wherein the one or more natural polymers comprises collagen.

42. A method for forming a tissue scaffold comprising lyophilising a synthetic polymer matrix, that has been contacted with one or more natural polymers selected from elastin, fibrin and collagen.

43. A method according to any of clauses 21 to 42, wherein the matrix is, or has been, contacted with the one or more natural polymers, in the absence of any cells.

44. A method according to any of clauses 21 to 43, wherein the scaffold is formed in vitro or ex vivo.

45. A method according to any of clauses 21 to 44 comprising seeding the matrix that has been contacted with the one or more natural polymers, with one or more cells, ex viva

46. A method according to clause 45, wherein the one or more cells comprises a stem cell, such as a hADSC.

47. A tissue scaffold obtained or obtainable by a method as defined in any of clauses 22 to 46.

48. A tissue or cell culture comprising a tissue scaffold as defined in any of clauses 1 to 20, or 47.

49. A tissue scaffold according to any of clauses 1 to 20, or 47, for use as a medicament.

50. A tissue scaffold according to any of clauses 1 to 20, or 47, for use in promoting tissue healing, regeneration or repair.

51. A method for promoting tissue regeneration, tissue healing or tissue repair comprising applying a tissue scaffold according to any of clauses 1 to 20, or 47, to a patient in need thereof.

52. Use of a tissue scaffold according to any of clauses 1 to 20, or 47, in the manufacture of a medicament for promoting tissue regeneration, tissue healing or tissue repair.

53. A tissue scaffold according to any of clauses 1 to 20, or 0.47, which is sterile.

54. A tissue scaffold according to any of clauses 1 to 20, or 47, which is packaged.

Specific examples of tissue scaffolds and methods for their production are provided, with reference to the following figures in which:

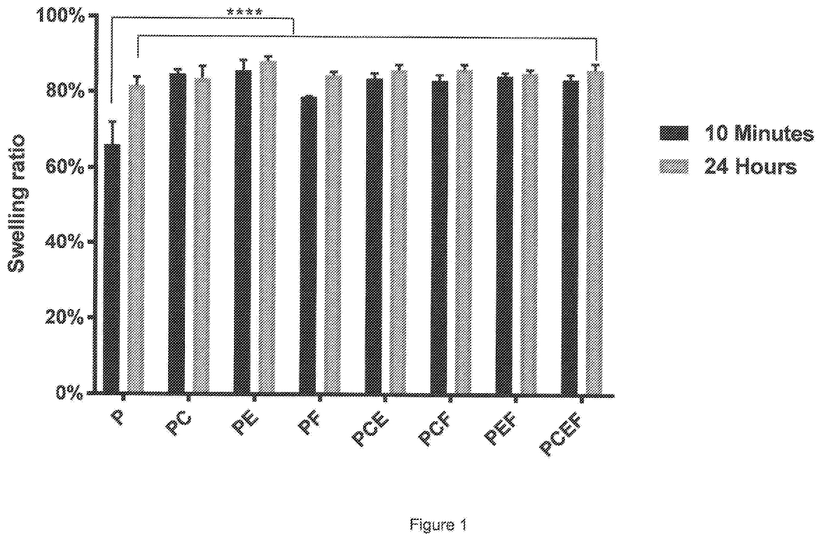

FIG. 1 shows swelling ratios of PCL-based composite scaffolds (**** p<0.0001 significance to P for 10 minutes and 24 hours);

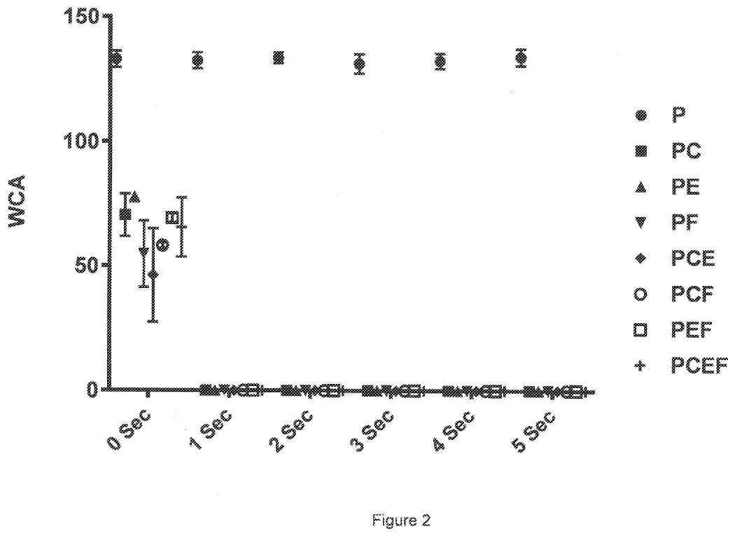

FIG. 2 shows water contact angle between PCL and other 7 PCL-based composite scaffolds;

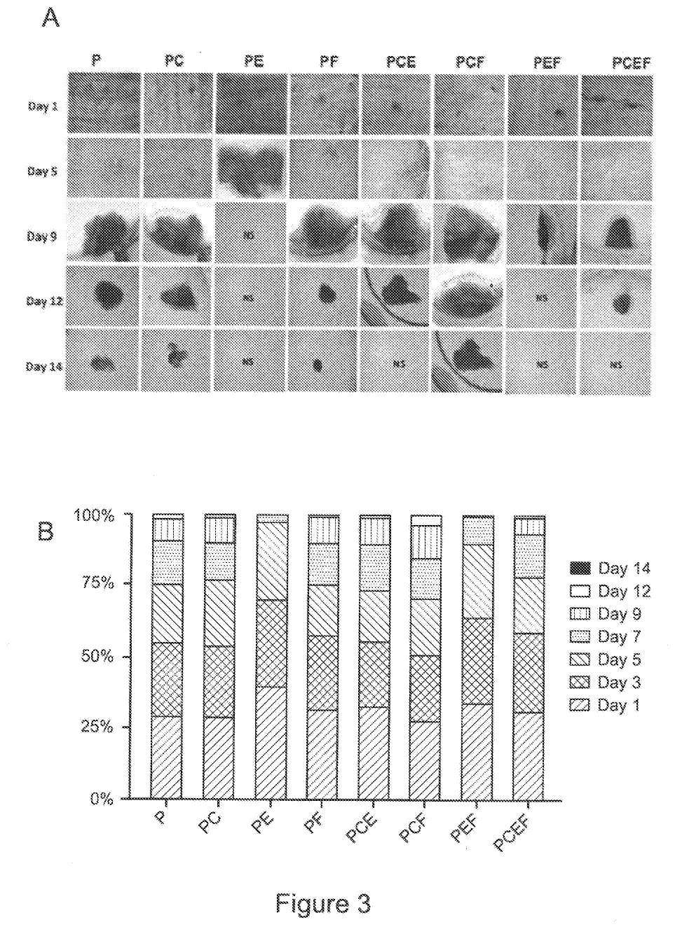

FIG. 3 shows accelerated degradation assay macroscopic images (A) and degradation rate (B) of PCL-based composite scaffolds;

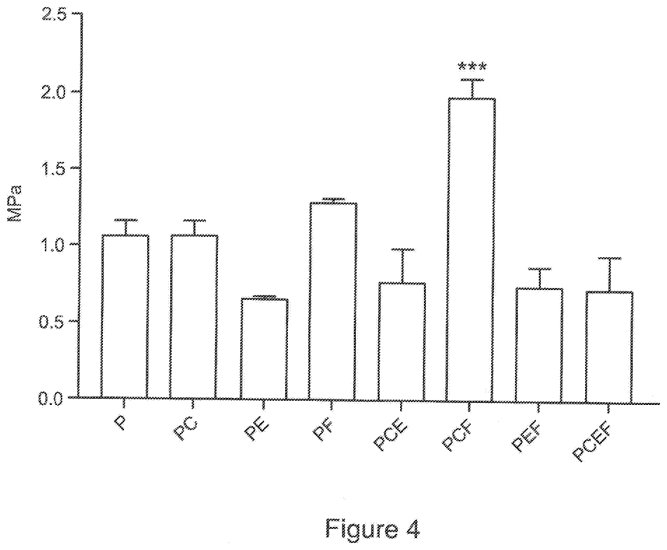

FIG. 4 shows mechanical properties for PCL-based composite scaffolds (*** p<0.001 significance to P);

FIG. 5 shows SEM images for PCL-based composite scaffolds (P(A), PC (B), PE(C), PF(D), PCE(E), PCF(F), PEF(G), PCEF(H)(+collagen, * elastin and #fibrin on the scaffold). Pore size distribution (I) and porosity for all scaffolds(J));

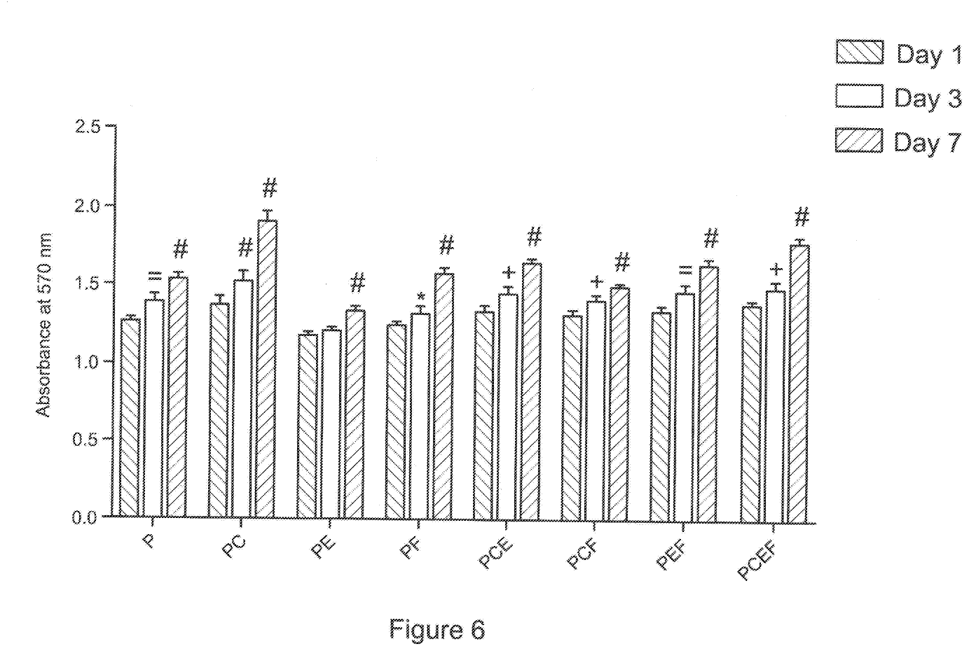

FIG. 6 shows Alamar blue absorption for PCL-based composite scaffolds at 570 nm for day 1, 3 and 7. *,+,=,# denotes statistical significance of p<0.05, p<0.01, =p<0.001, p<0.0001 respectively from day 1);

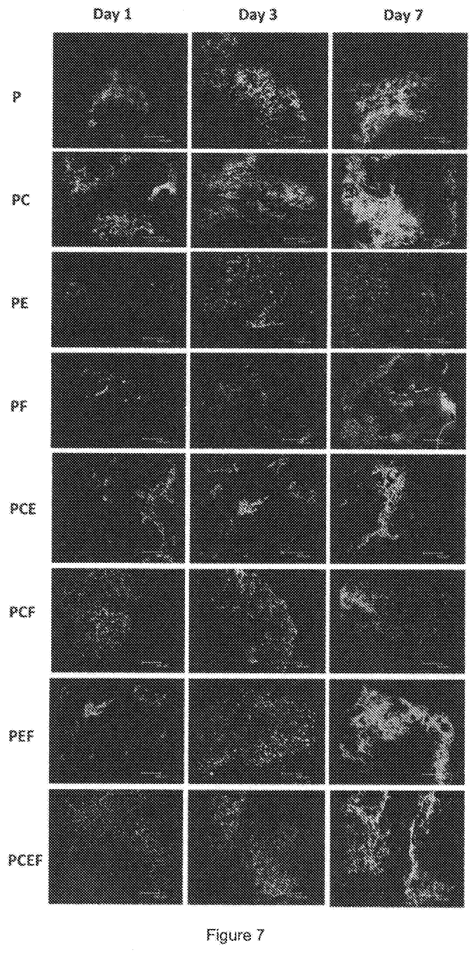

FIG. 7 shows a live/dead assay and cellular behaviour of PCL-based composites for day 1, 3 and 7;

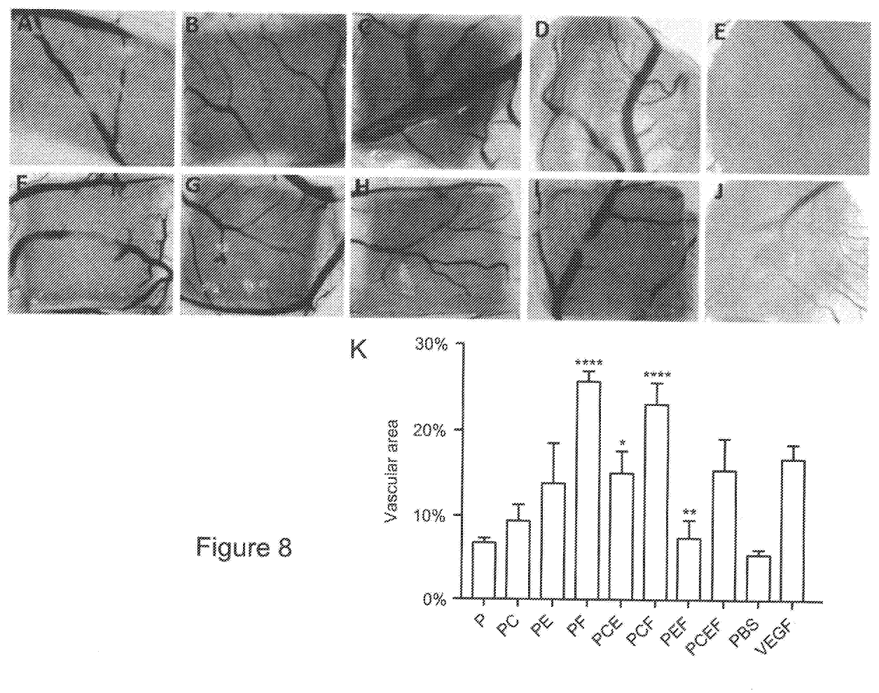

FIG. 8 shows P(A), PC (B), PE(C), PF(D), PBS, negative control (E), PCE (F) PCF(G), PEF (H) PCEF, (I), VEGF positive control (J) and vascular area percentages for each scaffold (K)(* p<0.05, ** p<0.01, **** p<0.0001 significance to P); and

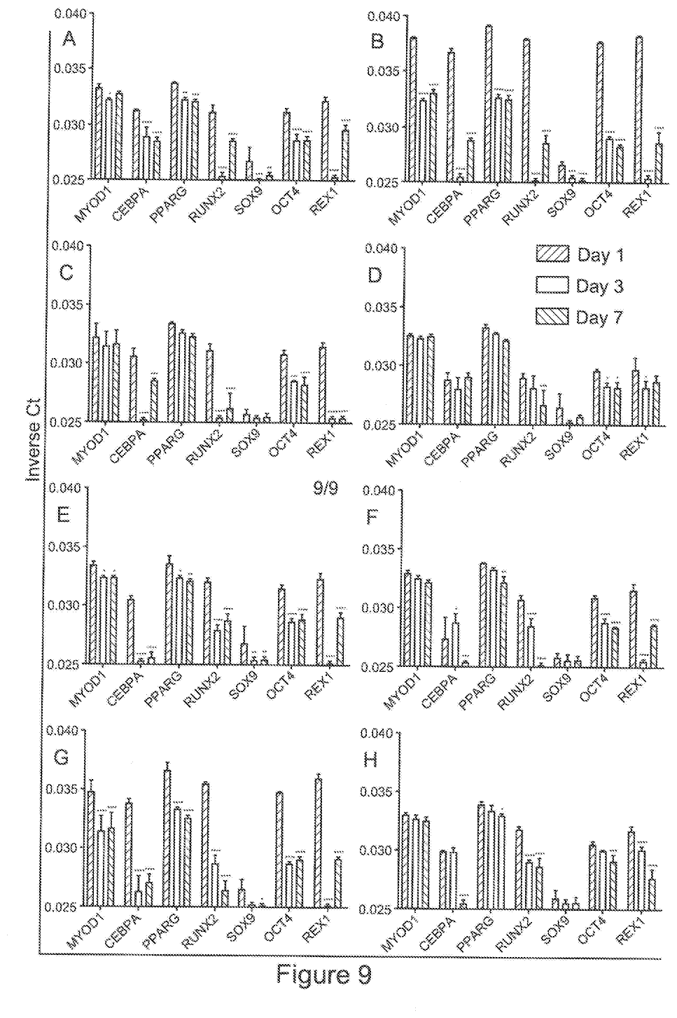

FIG. 9 shows a Differentiation pathway study of PCL-based composite scaffolds P(A), PC (B), PE(C), PF(D), PCE (E), PCF(F), PEF (G), PCEF (H). (* p<0.05, ** p<0.01*** p<0.001 and **** p<0.0001 to day 1 and # to day 3).

### EXAMPLES

### Example 1—Formulation of Natural Polymers

Collagen:—Collagen solution was made by using 90% rat tail collagen (First Link, Birmingham, UK) that was mixed with 10% 10× Minimal Essential Medium (Invitrogen, Paisley, UK) and neutralised by adding 5M and 1M NaOH drop-wise. (14)

Elastin:—Elastin solution was made by mixing 100 mg of insoluble elastin (Sigma, UK) with 1 ml of 0.5M oxalic acid (C₂H₂O₄) (freshly prepared) at room temperature.

Fibrin:—Fibrin solution was formed by dissolving 2% fibrinogen (Sigma, UK) in 1 ml of PBS (Gibco, UK) with thrombin (Sigma, UK).

### Example 2—Fabrication of PCL-Natural Polymer Composite Scaffolds

A custom electrospun PCL mimetix (Electrospinning company Ltd, Didcot, UK) air sheet of 1.1±0.04 mm membrane thickness was used in this study. A segment of 6×6 cm² was cut and coated by dipping into solution of i) collagen (PC), ii) elastin (PE), iii) fibrin (PF), iv) collagen/elastin (1:1) (PCE), v) collagen/fibrin (1:1) (PCF), vi) elastin/fibrin (1:1) (PEF), and vii) collagen/elastin/fibrin (1:1:1) (PCEF). All coated and control samples were lyophilised for 48 hours to form PCL-natural polymers composite scaffolds.

### Example 3—Swelling Ratio

The swelling ratio (SR) of the PCL and 7 composite scaffolds was measured from the dry mass and wet mass with the equation given below:

$$SR = \frac{{M}_{w} - {M}_{d}}{{M}_{w}} \times 100$$

where M_{d} is the dry mass and M_{w} is the wet mass of the scaffold. (15)

The wet mass of the scaffold was acquired by immersing the sample into 2 ml of distilled water (ELGA PURELAB Option), which was followed by measuring the scaffold mass with the digital scale (XS205 Mettler Toledo®). The SR of scaffolds immersed into distilled water for 10 minutes at room temperature and 24 hours at 37° C. and 5% CO₂ was calculated using the equation.

Scaffolds placed in water as an aqueous solvent absorbed excerpt of solvent, subsequently causing swelling of the scaffold. In this steady state, the swelling ratio of scaffolds is directly proportional to the degree of cross-linking of coating polymers to PCL. The swelling ratio for PCL after 10 minutes at room temperature was 66.02±5.94%, and it significantly increased to 81.62±2.23% at 37° C. after 24 hours. When PCL was coated with natural polymers, the swelling ratio was significantly higher (p<0.0001) in the range of 78.73% to 85.68% at 10 minutes of interaction with solvent (FIG. 1). When scaffolds were placed in the incubator at 37° C. with 5% CO₂ for 24 hours, the calculated swelling ratio was in the range of 81.60% to 88.19%. This change in the swelling ratio was marginal compared to 10 minutes except for PCL because of its hydrophobicity. The degree of swelling indicates an interaction between the scaffolds and the aqueous medium, and it is the result of solvent trying to dilute the scaffolds by penetration. However, this entropic increase was not affected by variation in temperature, time and pressure.

### Example 4—Dynamic Water Contact Angle

The wettability of the scaffolds was investigated using dynamic water contact angle (dWCA) measurements. The dWCA of each scaffold was determined with an experimental setup where a 30 μL distilled water droplet was dispensed onto each scaffold, while alterations to the dWCA were recorded as a video. (16) The dWCA were measured using screenshots taken at each second over the time interval of 0 to 5 seconds. The time point of 0 seconds was considered the initial contact of the water droplet with a developed profile on the scaffold. The measurements for dWCA on each scaffold were carried out manually with the aid of lmageJ (1.8.0_112) software. (NIH, US). The dWCA was measured using the principles of the Young's equation, where the angle was measured using the angle tool at 400% magnification from the water-scaffold interface to the line tangent to the perimeter of the water droplet. Water droplets absorbed into the scaffold within a millisecond and without a clearly defined profile were considered to have zero dWCA.

The dWCA indicates water-material interaction: a higher dWCA is directly proportional to a weaker interaction between water and the material. The dWCA for PCL was β2.71±3.28°, and after 5 seconds it was β4.04±3.40°, demonstrating the hydrophobic nature of PCL. However, when PCL was coated with natural polymers, dWCA decreased (PC 70.45±8.15°, PE 77.85±2.11°, PF 54.75±13.33°, PCE 46.27±18.74°, PCF 58.28±1.06°, PEF 69.28±1.71°, PCEF 65.50±11.95°) at time 0. At 1 second, the dWCA dropped to 0° indicating complete wettability of the coated scaffolds (FIG. 2). Among all scaffolds, PCE scaffold was the most hydrophilic, while PCL was the least.

### Example 5—In Vitro Accelerated Degradation Assay

The initial weights of the different scaffolds were measured using the X5205 Mettler Toledo® digital scale. The scaffolds were placed in 24 well-plate with 1M sodium hydroxide (NaOH) solution (Sigma-Aldrich, UK) and incubated at 37° C. At each time point, scaffolds were washed in distilled water (ELGA PURELAB Option) and lyophilised. The post-degradation weights of each scaffold were measured for each time point, and both microscopic and macroscopic images were obtained with a stereo microscope (0.7× [zoom], GX microscopes) and digital camera (Canon PowerShot SX400 IS).

In vitro accelerated degradation results indicated that all scaffolds degraded at a different rate. A change in the net weight of each biomaterial pre and post degradation was measured as one of the parameters of the degradation (FIG. 3B). A decrease in the net weight was observed for all the scaffolds from day 1. By day 5, the most degraded scaffold was PE (95%), and PCF was the least (70%), but P, PC, PF, PCE, PEF, PCEF were degraded in the range of 70%-75%. By day 7 PE was degraded 100% followed by PEF 98%, but PCF was still the least degraded (85%) scaffold. However, by the end of day 12, except P, PC, PF, PCF, the rest of the scaffolds were 100% degraded, and till day 14 PCF (1.16%), P (1.05%) and PC (0.02%) were marginally present. The gross observation revealed time-dependent morphological changes to the initial organised geometry (FIG. 3A).

### Example 6—Mechanical Properties

All PCL-based scaffolds were tested to failure using Zwick/Roell (Z005 model Ulm, Germany) with a test speed of 30 mm/min, Area input was calculated, and force was applied till failure. Break strength output was generated by testXpert V10.0 software (Zwick, GmbH & Co, Ulm Germany).

The mechanical properties of scaffolds were studied to analyse the effect of coating PCL with natural polymers. The Young's modulus for P (1.06±0.10 MPa) was similar to that of PC (1.06±0.08 MPa). On the other hand, Young's modulus decreased for other scaffolds (PE 0.66±0.01 MPa, PCE 0.77±0.21 MPa, PEF 0.74±0.12 MPa, PCEF 0.73±0.20 MPa), whilst it increased for PF (1.28±0.02 MPa) (FIG. 4), although this difference was marginal (p>0.05) (FIG. 4). However, PCF was significantly (P<0.05) the strongest (1.99±0.11 MPa) scaffold among all polymers. This demonstrates that coating of PCL with natural polymers influences the mechanical integrity of PCL

### Example 7—Scanning Electron Microscopy (SEM)

All PCL based composites were washed with deionised water to remove excess salts and unattached polymers. All samples were routinely prepared with a standard protocol and mounted on stubs following sputter-coating with carbon coater. All images were obtained using a secondary electron detector in a Philips XL 30 Field Emission SEM, operated at 5 kV, and average working distance was 10 mm. To measure porosity percentages and pore diameter range of scaffolds, all SEM images were quantitatively analysed using Images' bundled with 64-bit Java 1.6.0 (NIH, USA). A threshold frequency was adjusted to visualise all pores. An area fraction function was used for calculating porosity and by particle analysis function was used to determine the diameter of each.

All the scaffolds showed a variation in the pore properties after coating with the polymers. As expected, the most porous scaffold (FIG. 5J) was P (80.33%), with almost 75% of pore diameter distribution between 100 μm and 201+μm (FIG. 5I). These pore properties decreased when PCL was coated with the natural polymers: for PC (57.16% porous) 80% of pores were in the 1-100 μm range, for PE (64.78% porous) 85% of pores were in the range of 1-100 μm, and for PF (52.16% porous) 95% pores were in the range of 1-150 μm. However, pore properties were further altered when a binary and a ternary layer of natural polymer was added to the PCL scaffolds: the calculated pore distribution percentages for PCE (51.18% porous), PEF (50.41% porous) and PCEF (47.46% porous) had 90% of the pores in the range of 1-100 μm (FIG. 5). Among all scaffolds, porosity for PCF (22.86%) was the lowest with 97% of pores in the range of 0-50 μm.

### Example 8—Cell Culture

To evaluate the efficacy and biological activity of the scaffolds, human adipose-derived stem cells (hADSCs) (ATCC,UK) were cultured under standard culture conditions i.e. incubation at 37° C. with 5% CO₂ in MesenPRO RS™ basal cell culture medium (ThermoFisher, UK) supplemented with 2% MesenPRO RS™ growth supplement (ThermoFisher, UK) and 1% penicillin/streptomycin (Sigma-Aldrich, UK). Cells were passaged routinely at 80% confluency and all experiments were carried out at passage 3 or 4.

### Example 9—Cell Proliferation and Viability Assay

A resazurin based Alamar blue assay was performed for reduction power of cells as a quantitative measure of proliferation. In total 10⁵ cells were seeded on PCL-composite scaffolds and cultured for 1, 3 and 7 days. A 1:10 (vol/vol) AlamarBlue® (BIO-RAD, UK) reagent was added directly to the cells and incubated at 37° C. with 5% CO₂ for 3 hours. Absorbance was measured at 570 nm and 600 nm as reference wavelengths by using double beam UV/visible spectrophotometer (Spectronic Camspec Ltd, Garforth, UK). For cell viability a double staining kit (Invitrogen, UK) was utilised for florescence based staining of viable and dead cells. A solution of 0.2% (VN) calcein-AM and 0.1% (VN) propidium iodide (PI) were prepared in PBS. This assay solution was added to the scaffolds and incubated at 37° C. with 5% CO₂ for 30 minutes, Live and dead cells were visualised by fluorescence imaging and confocal microscopy (Leica DM IRE2 confocal microscope).

The results demonstrated continual viability and proliferation of the cells on all scaffolds (FIGS. 6 and 7). Alamar blue absorbance at 570 nm for all scaffolds on day 1 was in the range of 1.17-1.39 (FIG. 6). Cell attachment in PE and PF were lower than other scaffolds and cells showed a non-aggregated morphology. This can be correlated to Alamar blue absorbance (1.17±0.02 PE, 1.24±0.03 PF). Cell morphology for P, PC, PCE, PCF, PEF, PCEF was spindle-like and elongated and showed significant proliferative activity as compared to PC (1.37±0.04), PCE (1.34±0.03), PCF (1.31±0.1), PEF (1.34±1.04), and PCEF (1.38±0.5). By day 3 all the scaffolds showed increase in the absorbance with the exception of PE which exhibited the lowest absorbance (1.21±0.02). PC displayed the highest absorbance (1.52±0.06) while the absorbance in other scaffolds (P, PF, PCE, PCF, PEF and PCEF) was in the range of 1.31-1.49. Cell morphology for PE, PEF and PCEF was distinctive and non-aggregated, but cells on PC showed aggregated morphology (FIG. 7). By day 7, cells on scaffolds such as P, PC, PF, PCE, PEF and PCEF showed aggregated morphology. Cells on PE, PCF scaffolds showed distinctive spindle-shaped morphology. Among all scaffolds, cells on PC proliferated the most and those on PE proliferated the least.

### Example 10—Angiogenetic Response

Fertilised pathogen free eggs were obtained from a commercial supplier and incubated at 38° C. with 40-45% humidity for 3 days. On embryonic day 3 (ED 3) eggs were cracked into a glass bowl set up (17). An ex ovo culture was maintained at 37.5° C. with 3% CO₂ and an average humidity in the range of 80-85%. At ED 9 all scaffolds and controls (filter discs dipped in PBS as a negative control and VEGF was a positive control) were implanted on the developing chorio-allantoic membrane (CAM) to allow infiltration of blood vessels. Culture was maintained up to ED 13 where embryos were euthanised as per UK Home Office regulations. The CAM was fixed in 4% paraformaldehyde for 15 minutes (to avoid bleeding after excision) and scaffolds were excised. All macroscopic images were obtained by stereo microscopy (0.7× [zoom], GX microscopes) and analysed using ImageJ software. A vascular area percentage was calculated for each scaffold using binary image and area fraction function.

To investigate the angiogenic response, all scaffolds were excised from the CAM on ED13 and vasculature was studied for each scaffold (FIG. 8A-J). Vascular area for P (6.86±0.54%) was the lowest among all scaffolds and was characterised by a single vessel, with majority of the area remaining non-vascularised. PC scaffolds (9.62±1.87%) had two large vessels with a number of capillary plexus. The vascular invasion for PF (25.94±1.17%) was the highest among all scaffolds with a cascade of capillaries with major vessels. PCE (15.09±2.63%) displayed infiltration of major vessels with the minimum capillary plexus. In PE (13.86±4.73%) anastomosis was present but PEF (7.59±2.10%) had low vasculature. On the other hand, PCF (23.13±2.56) had a high vasculature with three major vessels and numerous capillary plexus. The vasculature for PCEF was 15.70±3.47% with a major vessel and thick capillaries. These results were compared to the vascular area of PBS (5.68±0.48%) which was the negative control and VEGF (16.89±1.65%) which is a positive control (FIG. 8K).

### Example 11—Differentiation Study

RNA was isolated by TRIzol (Invitrogen, Paisley, UK) method on days 1, 3 and 7 of culture and yield was quantified by spectrophotometry (Spectronic Camspec Ltd, Garforth, UK). A cDNA synthesis was carried out using Precision nanoscript 2 reverse transcription kit (Primer Design, Southampton, UK) and quantitative PCR was performed with custom designed and synthesised primers (Table1) (Primer design, Southampton, UK) for analysis of differentiation pathways of hADSC. Gene expression was expressed as the inverse of Ct values. Beta-2-Microglobulin (B2M), Glyceraldehyde 3-phosphate dehydrogenase (GAPDH) and Peptidylprolyl Isomerase A (PPIA) were used as reference genes.

TABLE 1
list of primers

| Name | | | Accession |
|---|---|---|---|
| of gene | Forward primer | Reverse primer | number |
| MYOD1 | CGCCTGAGCAAAG | GCCCTCGATATAGCG | NM_002478 |
| | TAAATGAG | GATG | |
| | (SEQ ID NO: 1) | (SEQ ID NO: 2) | |
| PPARG | GAATAAAGATGGG | AACTTCAGCAAACTC | NM_138711 |
| | GTTCTCATATCC | AAACTT | |
| | (SEQ ID NO: 3) | (SEQ ID NO: 4) | |
| CEBPA | CGGCAACTCTAGT | CAAATAAAATGACAA | NM_004364 |
| | ATTTAGGATAAC | GGCACGATT | |
| | (SEQ ID NO: 5) | (SEQ ID NO: 6) | |
| RUNX2 | TTCTCCCCTTTTC | CAAACGCAATCACTA | NM_004348 |
| | CCACTGA | TCTATACCAT | |
| | (SEQ ID NO: 7) | (SEQ ID NO: 8) | |
| SOX9 | GGACCAGTACC | AATCCGGGTGGTCCT | NM_004348 |
| | GCACTTG | TCTTG | |
| | (SEQ ID NO: 9) | (SEQ ID NO: 10) | |
| OCT4 | CACTAAGGAAGGA | GGGATTAAAATCAAG | NM_002701 |
| | ATTGGGAACA | AGCATCATTG | |
| | (SEQ ID NO: 11) | (SEQ ID NO: 12) | |
| REX1 | CGTTTCGTGTCCC | CCTCTTGTTCATTCTT | NM_174900 |
| | TTTCA | GTTCGTATT | |
| | (SEQ ID NO: 13) | (SEQ ID NO: 14) | |

Gene expression of MSC markers (OCT4 and REX1) and mesenchymal lineage specific differentiation markers —adipogenic (CEBPA and PPARG), osteogenic (RUNX2), myogenic (MYOD1) and chondrogenic (SOX9)- were studied in cell-seeded scaffolds. hADSCs seeded on tissue culture plastic was used as a control. It was noticed that the Ct values of the three housekeeping genes used (GAPDH, B2M and PPIA) showed significant fluctuation between the three time points. Hence, the relative expression of candidate genes could not be calculated accurately due to the lack of an ideal reference gene. Alternatively, results have been represented as the inverse of Ct values. Ct values ranging from 25 to 40 will correspond to inverse Ct values 0.04 to 0.025 respectively.

Scaffold wise gene expression profiles have been illustrated in FIG. 9. All genes were observed to exhibit highest expression on day 1. The overall range of expression of all genes (excluding SOX9) on day 1 was observed to be the highest in P (0.037-0.039) (FIG. 9A), closely followed by PCEF (0.034-0.037) (FIG. 9H). Among the remaining scaffolds PC (FIG. 9B), PF (FIG. 9D), PCF (FIG. 9F) and PEF (FIG. 9G) showed moderate expression in the range 0.030-0.034 on day 1. Interestingly, PE (FIG. 9C) and PCE (FIG. 9E) exhibited the lowest range of expression (0.027-0.033), where all the genes except CEBPA (0.029 in PE and 0.027 in PCE) showed an expression greater than 0.030.

Myogenic lineage marker MYOD1 exhibited a significant downregulation on day 3 in P, PC, PCF and PCEF (p<0.05), but remained at the same level (0.031-0.033) at day 7. PE, PF, PCE and PEF did not show any significant difference in expression between time points and remained consistent at 0.031-0.032. Adipogenic lineage markers PPARG and CEBPA exhibited contrasting profiles. PPARG remained constant on day 3 and 7 at 0.032-0.033 in all scaffolds without exception. On the other hand, CEBPA was downregulated to negligible levels (0.025) at day 7 in PCF, PCE and PEF; but showed a significant upregulation (p<0.001) from negligible levels on day 3 (0.025) to 0.028 on day 7 in P and PF. Osteogenic lineage marker RUNX2 showed a progressive downregulation trend in PF, PE, PCE and PCEF. In P and PC it was downregulated to negligible levels (0.025, p<0.0001) on day 3, but showed a significant upregulation on day 7 (0.028. p<0.0001). it remained constant at 0.027-0.028 on day 3 and day 7 in PCF and PEF. Chondrogenic lineage marker SOX9 presented overall negligible expression at all time points (0.026-0.025). OCT4, which is responsible for maintenance of self-renewal potential of stem cells has displayed a consistent and steady downregulation pattern in all scaffolds. However, REX1 which is responsible for multi-potency of stem cells was downregulated completely on day 3 (0.025) but upregulated significantly on day 7 (0.028-0.029, p<0.0001) in the majority of scaffolds (P, PC, PCF, PCE and PCEF). Among the remaining scaffolds, it remained at 0.027-0.028 on day 7 (PE and PEF), except for PF where it was reduced to negligible levels on day 3 and day 7 (0.025).

### Discussion

The inventors have appreciated that synthetic polymers, such as PCL can be combined with natural polymers that are present in the ECM of human tissues such as collagen, fibrin and elastin, to form scaffolds. Surprisingly, the inventors have found that certain advantages of both synthetic and natural polymers can be retained, whilst certain disadvantages can be negated. This concept may be considered to be a ‘symbiotic relationship’.

Any material with a dWCA>90 is considered to be hydrophobic. It is a well-established that synthetic polymers, such as PCL, are hydrophobic, which can result in weaker cell attachment. This can be improved by treating PCL with plasma, UV, chemical coating, or radiation (8), but ut these treatments demonstrate weaker water binding affinity which may affect PCL's physical and biological activities (18). To address this issue, the inventors have coated PCL with natural polymers and the resulting scaffolds exhibited strong affinity towards liquid by transforming surface chemistry from hydrophobic to hydrophilic, as demonstrated by dWCA=0° within 1 second. In all composite scaffolds there was high cohesion towards water, hence no residual water droplet was present, and scaffolds gained complete wettability and a change in properties from hydrophobic to hydrophilic. This concept of liquid cohesion and water affinity is described in Young's essay. (19, 20)

Biodegradation rate of PCL is inferior to other polylactides because it has frequent ester bonds, which impede degradation of PCL up to 2-4 years by hydrolytic degradation of these ester groups (21). Ideally, the degradation rate of PCL should be synchronised with neo-tissue formation because a slower degradation rate hinders cell proliferation and tissue repair, whilst faster degradation results in impaired tissue formation (22-25). Therefore, it has not been considered feasible to use PCL in all tissue regeneration applications, because the competence level of every tissue is different. Surprisingly, in this study the inventors have demonstrated that by coating PCL with natural polymers they can tailor the degradation of scaffolds corresponding to the regenerative power of the native tissues, e.g. PE, PEF could be used for large organ engineering like skin and liver which have strong regeneration/repair abilities in contrast to scaffolds such as P, PC, PF, PCE, PCEF that can be used for soft connecting tissue engineering such as tendon and ligaments which have weaker regeneration properties (26).

Diffusion of oxygen in tissues is limited to 100-200 mm. Therefore, to regenerate a neo-tissue beyond that, a cascade of blood vessels is required (27). Hence, a scaffold requires an ideal pore size range for angiogenesis to ensure the flow of nutrients/waste products and cellular migration. CAM assay acted as an ex vivo bioreactor to study angiogenesis on the scaffolds. An ideal pore size for the angiogenesis is still unclear in the literature, but the majority of studies suggest essentially higher pore size and pore ranges for angiogenesis. In our study, although P was the most porous (80.33%) and largest pore-sized (75% pores between 100-200 μm) scaffold, it had inadequate surface topological properties to attract a vascular network (6.86%). However, by converting to hydrophilic by post-coating with natural polymers, it was surprisingly found to attract a strong vascular invasion onto the scaffold. It should be noticed that fibrin coated scaffolds had a maximum outgrowth of new blood vessels from the existing vasculature, such as PF, which had a medium porosity (52.16%), 80% pores in the range 1-100 μm and the highest vascular network area (25.94%). Also, a less porous scaffold-like PCF (22.86%) with 97% pores in the range 0-50 μm had a high vascular network (23.13%). Combination of just fibrin or fibrin and collagen with PCL proved to be pro-angiogenic, elastin by itself or with collagen is moderately pro-angiogenic, but the combination of elastin and fibrin is not very pro-angiogenic (e.g. PEF). This can be correlated to the previous finding about the pro-angiogenic effect of fibrin and fibrin-collagen composites (28, 29). This demonstrates that in the initial stages of tissue repair not only pore size and porosity but also the material topological properties and surface chemistry play a vital role in the formation of a vascular network.

The microstructure of scaffolds reflects the dynamic interaction between cells and ECM. Hence, the mechanical properties of scaffolds should be adequate to the anatomical site until neo-tissue is formed (30). However, a large pore size will compromise the mechanical integrity of the scaffold due to the large void volume, while small pores-based scaffolds will be mechanically stronger (31). This can be correlated to the enhanced mechanical properties of PCF which has 97% of the pores in the range of 0-50 μm. Scaffolds such as PCF have the potential for orthopaedic tissue engineering as they are mechanically stronger tissues, and scaffolds like PE, PCE, PEF, PCEF could be candidates for soft tissue engineering.

Alongside investigating the material properties and basic cell-material interactions, the differentiation potential of PCL and its composites with natural ECM components were evaluated. Differentiation of hADSCs seeded on PCL scaffolds is affected by a combination of factors such as growth factor/chemical stimuli, surface topography, mechanical properties, material porosity and pore size gradient (16, 32-34).

The majority of differentiation studies that are performed on 3D scaffolds seeded with hADSC are carried out with external chemical stimulus such as specific differentiation medium. In contrast, this study evaluated the differentiation pathway of hADSC induced by material properties as a physical stimulus. The markers for hADSC main stream lineage tendencies, i.e. adipogenic, osteogenic, chondrogenic and myogenic were screened. The most common housekeeping gene GAPDH was originally used as a reference, but it showed a very high fluctuation and variability in expression across the different scaffolds. This observation correlates with the report that compared 8 common housekeeping genes for their stability and reliability in mesenchymal stem cells. According to this study, B2M and PPIA were the most stable reference genes. (35) Nevertheless, these two references displayed similar behaviour to GAPDH This could be explained by the findings that gene expression of the same cells vary between monolayered and 3D cultures, subsequently influencing their morphology and functionality. (36) Although hADSC are considered to exhibit stable expression of B2M and PPIA in monolayer culture, there are contradicting reports about hADSC seeded on 3D scaffolds. TATA-box protein that was claimed to be the least stable in monolayer culture of MSCs, (35) has been demonstrated to be the most stable among 31 reference genes screened in bone marrow derived MSC seeded on 3D scaffolds.(37) This necessitates the screening of reference genes in hADSCs seeded PCL and their composite scaffolds individually, which is not feasible as each scaffold will have its own stable housekeeping gene making it incomparable to other scaffolds in the study. Hence, the relative expression of the genes of interest could not be represented as the gold standard (ddCt/fold change). Therefore, the inventors are reporting a novel method of representing gene expression using inverse Ct, as the Ct values are inversely proportional to gene expression. This method of representation provides a measure of the general trend of upregulation or downregulation in multiple scaffold comparisons. Although it is not an absolute measure, it facilitates the determination of general inclination towards a particular lineage.

All the genes exhibited high expression on day 1 in all scaffolds. This could be due to the sudden change in the architectural environment of the cells leading to a surge in these genes expression to enable cells to accustom to the new environment (38). In the absence of external chemical stimuli, the cells cannot be expected to pursue further differentiation. However, the subsequent persistence or downregulation of lineage markers on day 3 and day 7 suggests that the cells are tending towards particular lineages over the others (39). All scaffolds showed persistent expression of MYOD1 (>0.030), suggesting that all PCL-based scaffolds are ideal candidates for myogenic differentiation. Indeed, the majority of pores in the composite scaffolds has a size <105 μm which is crucial for myogenic differentiation.(40)

Pore sizes between 500 μm-850 μm induced osteogenic response in MSCs, (41) but in the inventor's studies P, PC, PCF and PEF showed significant expression of osteogenic marker RUNX2 on day 7, although pore sizes of these scaffolds are <500 μm. This suggests that apart from pore size, scaffold composition also influences osteogenic differentiation. Expression of chondrogenic marker SOX9 is negligible in all scaffolds at all three time points owing to the sub-optimal pore size of >300 μm, as a mean pore size of 300 μm stimulates chondrogenic gene expression and cartilage-like matrix deposition as compared with smaller pore sizes (94 μm, β0 μm). (39, 42). In terms of adipogenic markers, PPARG was consistently expressed in all scaffolds by the end of day 7, but

CEBPA expression was downregulated at day 3. However, at day 7, significant upregulation was observed in comparison to day 3 (except PCF and PCEF). PPARG is a well established adipogenic marker and is essential for activating CEBPA induced adipogenesis. PPARG has been reported to successfully induce adipogenic differentiaiton in the absence of CEBPA, while CEBPA is not pro-adipogenic in the absence of PPARG (43, 44). In addition, CEBPA is reported to be expresed late in adipogenesis, i.e. it is not expressed until pre-adipocyte stage (45) and is exclusive for development of white adipose tissue. This explains the absence or lower levels of CEBPA in scaffolds in comparison to consistently expressed PPARG. This suggests that PCL-based composites could be beneficial for adipogenesis. (46)

In P and all collagen containing scaffolds, the multipotency marker, REX1 is showing an upregulation trend on day 7. On the other hand, OCT4, which is a critical transcription factor that ensures self-renewal of stem cells, is invariably downregulated in all scaffolds by day 7. This suggests a gradual reduction in sternness of ADSCs, while still maintaining the multipotency feature as the cells are still undifferentiated or beginning to travel towards differentiation.

The ‘symbiotic relationship’ between natural and synthetic polymers, such as PCL, facilitates an economically and technically efficient method for tailoring scaffold's properties such as mechanical strength, porosity, wettability, angiogenesis potential, cell-material interaction, etc, which are crucial for tissue and organ regeneration. In addition, these scaffolds have demonstrated lineage specific differentiation potential. This provides substantial evidence that prolonged culture of ADSCs in these scaffolds can lead to effective cell differentiation even in the absence of external stimuli like mechanical or chemical induction.

### REFERENCES

1. Hollister S J. Porous scaffold design for tissue engineering. Nature materials. 2005; 4(7):518-24.
2. Yoon D M, Fisher J P. Natural and Synthetic Polymeric Scaffolds. In: Narayan R, editor. Biomedical Materials. Boston, Mass.: Springer U S; 2009. p. 415-42.
3. Davison N L, Barrère-de Groot F, Grijpma D W. Chapter 6—Degradation of Biomaterials. Tissue Engineering (Second Edition). Oxford: Academic Press; 2014. p. 177-215.
4. Bhatia S. Natural Polymers vs Synthetic Polymer. In: Bhatia S, editor. Natural Polymer Drug Delivery Systems: Nanoparticles, Plants, and Algae. Cham: Springer International Publishing; 2016. p. 95-118.
5. Gunatillake P, Mayadunne R, Adhikari R. Recent developments in biodegradable synthetic polymers. Biotechnol Annu Rev. 2006; 12:301-47.
6. Gunatillake P A, Adhikari R. Biodegradable synthetic polymers for tissue engineering. Eur Cell Mater. 2003; 5:1-16; discussion
7. Hoskins J N, Grayson S M. Synthesis and Degradation Behavior of Cyclic Poly(ε-caprolactone). Macromolecules. 2009; 42(17):6406-13.
8. Cipitria A, Skelton A, Dargaville T R, Dalton P D, Hutmacher D W. Design, fabrication and characterization of PCL electrospun scaffolds-a review. Journal of Materials Chemistry. 2011; 21(26):9419-53.
9. Place E S, George J H, Williams C K, Stevens M M. Synthetic polymer scaffolds for tissue engineering. Chemical Society Reviews. 2009; 38(4):1139-51.
10. Howard D, Buttery L D, Shakesheff K M, Roberts S J. Tissue engineering: strategies, stem cells and scaffolds. Journal of Anatomy. 2008; 213(1):66-72.
11. O'Brien F J. Biomaterials & scaffolds for tissue engineering. Materials Today, 2011; 14(3):88-95.
12. Frese L, Dijkman P E, Hoerstrup S P. Adipose Tissue-Derived Stem Cells in Regenerative Medicine. Transfusion Medicine and Hemotherapy. 2016; 43(4):268-74.
13. Augello A, De Bari C. The regulation of differentiation in mesenchymal stem cells. Hum Gene Ther. 2010; 21(10):1226-38.
14. Brown R A, Wiseman M, Chuo C B, Cheema U, Nazhat S N. Ultrarapid Engineering of Biomimetic Materials and Tissues: Fabrication of Nano- and Microstructures by Plastic Compression. Advanced Functional Materials. 2005; 15(11):1762-70.
15. Eke G, Mangir N, Hasirci N, MacNeil S, Hasirci V. Development of a U V crosslinked biodegradable hydrogel containing adipose derived stem cells to promote vascularization for skin wounds and tissue engineering. Biomaterials. 2017; 129:188-98.
16. Fu W, Liu Z, Feng B, Hu R, He X, Wang H, et al. Electrospun gelatin/PCL and collagen/PLCL scaffolds for vascular tissue engineering. Int J Nanomedicine. 2014; 9:2335-44.
17. Auerbach R, Kubai L, Knighton D, Folkman J. A simple procedure for the long-term cultivation of chicken embryos. Developmental biology. 1974; 41(2):391-4.
18. Augustine R, Saha A, Jayachandran V P, Thomas S, Kalarikkal N. Dose-Dependent Effects of Gamma Irradiation on the Materials Properties and Cell Proliferation of Electrospun Polycaprolactone Tissue Engineering Scaffolds. International Journal of Polymeric Materials and Polymeric Biomaterials. 2015; 64(10):526-33.
19. Law K-Y. Definitions for Hydrophilicity, Hydrophobicity, and Superhydrophobicity: Getting the Basics Right. The Journal of Physical Chemistry Letters. 2014; 5(4):686-8.
20. Young T. III. An essay on the cohesion of fluids. Philosophical Transactions of the Royal Society of London. 1805; 95:65-87.
21. Woodruff M A, Hutmacher D W. The return of a forgotten polymer—Polycaprolactone in the 21st century. Progress in Polymer Science. 2010; 35(10):1217-56.
22. Yildirimer L, Seifalian A M. Three-dimensional biomaterial degradation—Material choice, design and extrinsic factor considerations. Biotechnol Adv. 2014; 32(5):984-99.
23. Raghunath J, Georgiou G, Armitage D, Nazhat S N, Sales K M, Butler P E, et al. Degradation studies on biodegradable nanocomposite based on polycaprolactone/polycarbonate (80:20%) polyhedral oligomeric silsesquioxane. Journal of Biomedical Materials Research Part A. 2009; 91A(3):834-44.
24. Lewandrowski K-U, Gresser J D, Wise D L, Trantolo D J, Hasirci V. Tissue responses to molecularly reinforced polylactide-co-glycolide implants. Journal of Biomaterials Science, Polymer Edition. 2000; 11(4):401-14.
25. Malikmammadov E, Tanir T E, Kiziltay A, Hasirci V, Hasirci N. PCL and PCL-based materials in biomedical applications. J Biomater Sci Polym Ed. 2017:1-31.
26. Yang G, Rothrauff B B, Tuan R S. Tendon and Ligament Regeneration and Repair: Clinical Relevance and Developmental Paradigm. Birth defects research Part C, Embryo today: reviews. 2013; 99(3):203-22.
27. Carmeliet P, Jain R K. Angiogenesis in cancer and other diseases. Nature. 2000; 407(6801):249-57.
28. Dvorak H F, Harvey V S, Estrella P, Brown L F, McDonagh J, Dvorak A M. Fibrin containing gels induce angiogenesis. Implications for tumor stroma generation and wound healing. Laboratory investigation; a journal of technical methods and pathology. 1987; 57(6):673-86.
29. Yang Y I, Seal D L, Kim H I, Cho M H, Lee S J. Composite fibrin and collagen scaffold to enhance tissue regeneration and angiogenesis. Current Applied Physics. 2007; 7:e103-e7.
30. Loh Q L, Choong C. Three-dimensional scaffolds for tissue engineering applications: role of porosity and pore size. Tissue engineering Part B, Reviews. 2013; 19(6):485-502.
31. Karageorgiou V, Kaplan D. Porosity of 3D biomaterial scaffolds and osteogenesis. Biomaterials. 2005; 26(27):5474-91.
32. Capkin M, Cakmak S, Kurt F O, Gumusderelioglu M, Sen B H, Turk B T, et al. Random/aligned electrospun PCL/PCL-collagen nanofibrous membranes: comparison of neural differentiation of rat AdMSCs and BMSCs. Biomedical Materials. 2012; 7(4).
33. Ruminski S, Ostrowska B, Jaroszewicz J, Skirecki T, Wlodarski K, Swieszkowski W, et al. Three-dimensional printed polycaprolactone-based scaffolds provide an advantageous environment for osteogenic differentiation of human adipose-derived stem cells. Journal of Tissue Engineering and Regenerative Medicine. 2018; 12(1):E473-E85.
34. Safaeijavan R, Soleimani M, Divsalar A, Eidi A, Ardeshirylajimi A. Comparison of random and aligned PCL nanofibrous electrospun scaffolds on cardiomyocyte differentiation of human adipose-derived stem cells. Iranian Journal of Basic Medical Sciences. 2014; 17(11):903-11.
35. Li X Y, Yang Q W, Bai J P, Xuan Y L, Wang Y M. Identification of appropriate reference genes for human mesenchymal stem cell analysis by quantitative real-time PCR. Biotechnology Letters. 2015; 37(1):67-73.
36. Frith J E, Thomson B, Genever P G. Dynamic Three-Dimensional Culture Methods Enhance Mesenchymal Stem Cell Properties and Increase Therapeutic Potential. Tissue Engineering Part C-Methods. 2010; 16(4):735-49.
37. Rauh J, Jacobi A, Stiehler M. Identification of Stable Reference Genes for Gene Expression Analysis of Three-Dimensional Cultivated Human Bone Marrow-Derived Mesenchymal Stromal Cells for Bone Tissue Engineering, Tissue Engineering Part C-Methods. 2015; 21(2):192-206.
38. Cao J, Luo Z, Cheng Q, Xu Q, Zhang Y, Wang F, et al. Three-dimensional regulation of transcription. Protein & Cell. 2015; 6(4):241-53.
39. Lien S-M, Ko L-Y, Huang T-J. Effect of pore size on ECM secretion and cell growth in gelatin scaffold for articular cartilage tissue engineering. Acta Biomaterialia, 2009; 5(2):670-9.
40. Kim T H, Oh S H, Kwon E B, Lee J Y, Lee J H. In vitro evaluation of osteogenesis and myogenesis from adipose-derived stem cells in a pore size gradient scaffold. Macromolecular Research. 2013; 21(8):878-85.
41. Vissers CAB, Harvestine J N, Leach J K. Pore size regulates mesenchymal stem cell response to Bioglass-loaded composite scaffolds. Journal of Materials Chemistry B. 2015; 3(44):8650-8.
42. Matsiko A, Gleeson J P, O'Brien F J. Scaffold mean pore size influences mesenchymal stem cell chondrogenic differentiation and matrix deposition. Tissue engineering Part A. 2015; 21(3-4):486-97.
43. Wu Z D, Rosen E D, Brun R, Hauser S, Adelmant G, Troy A E, et al. Cross-regulation of C/EBP alpha and PPAR gamma controls the transcriptional pathway of adipogenesis and insulin sensitivity. Molecular Cell. 1999; 3(2):151-8.
44. Rosen E D, Hsu C H, Wang X, Sakai S, Freeman M W, Gonzalez F J, et al. C/EBPalpha induces adipogenesis through PPARgamma: a unified pathway. Genes & development. 2002; 16(1):22-6.
45. Darlington G J, Ross S E, MacDougald O A. The role of C/EBP genes in adipocyte differentiation. Journal of Biological Chemistry. 1998; 273(46):30057-60.
46. Brannmark C, Paul A, Ribeiro D, Magnusson B, Brolen G, Enejder A, et al. Increased Adipogenesis of Human Adipose-Derived Stem Cells on Polycaprolactone Fiber Matrices. PLoS ONE. 2014; 9(11):e113620.

1. A tissue scaffold comprising a synthetic polymer and at least one natural polymer selected from fibrin, collagen and elastin, wherein:
a) the at least one natural polymer comprises fibrin and: i) elastin; and/or ii) collagen;
b) the synthetic polymer is in the form of a fibrous matrix and the matrix is coated with the at least one natural polymer; and/or
c) the at least one natural polymer comprises elastin, the elastin being unfractionated and comprising soluble elastin.

2. A tissue scaffold according to claim 1, wherein the at least one natural polymer comprises fibrin and: i) elastin; and/or ii) collagen.

3. A tissue scaffold according to claim 2, wherein the at least one natural polymer comprises fibrin and elastin.

4. A tissue scaffold according to any preceding claim, wherein the at least one natural polymer comprises elastin which is unfractionated and comprises soluble elastin.

5. A tissue scaffold according to any preceding claim, which is dry or substantially free of water.

6. A tissue scaffold according to any preceding claim, which is lyophilised.

7. A tissue scaffold according to any preceding claim, wherein the synthetic polymer is in the form of a matrix, optionally wherein the matrix is fibrous.

8. A tissue scaffold according to claim 7, wherein the matrix is coated with the, or each, natural polymer.

9. A tissue scaffold according to any preceding claim which comprises nanofibers, optionally wherein the synthetic polymer is in the form of a nanofibrous matrix.

10. A tissue scaffold according to any preceding claim, which comprises electrospun fibres, optionally wherein the synthetic polymer is in the form of an electrospun matrix.

11. A tissue scaffold according to any preceding claim, comprising fibrin, collagen and elastin.

12. A tissue scaffold according to any preceding claim, wherein the synthetic polymer comprises polycaprolactone (PCL).

13. A tissue scaffold according to any preceding claim, in combination with at least one cell.

14. A tissue scaffold according to claim β, wherein the at least once cell comprises a stem cell, optionally wherein the stem cell is hADSC.

15. A tissue scaffold according to any preceding claim, which is ex vivo or in vitro.

16. A method for forming a tissue scaffold having a synthetic polymer and one or more natural polymers selected from elastin, fibrin and collagen, comprising:
a) contacting the synthetic polymer with the one or more natural polymers, wherein the one or more natural polymers comprise fibrin and: i) elastin; and/or ii) collagen;
b) coating the synthetic polymer with the one or more natural polymers, wherein the synthetic polymer is in the form of a fibrous matrix; and/or
c) contacting the synthetic polymer with the one or more natural polymer, wherein the one or more natural polymers comprise elastin, and wherein the elastin is unfractionated and comprises soluble elastin.

17. A method according to claim 16, wherein the synthetic polymer is in the form of a matrix, optionally wherein the matrix is fibrous.

18. A method according to claim 17, wherein the matrix comprises nanofibers, wherein the polymer matrix has been formed by electrospinning, or wherein the method comprises electrospinning a solution comprising the synthetic polymer.

19. A method according to claim 17 or claim 18, comprising coating the matrix with the one or more natural polymers.

20. A method according to any of claims 17 to 19, comprising contacting the matrix with a solution or suspension comprising the one or more natural polymers.

21. A method according to claim 20, comprising immersing the matrix in a solution or suspension comprising the one or more natural polymers.

22. A method according to any of claims 16 to 21, comprising drying the synthetic polymer that has been contacted with the one or more natural polymers, preferably lyophilising the synthetic polymer that has been contacted with the one or more natural polymers.

23. A method according to any of claims 16 to 22, wherein the synthetic polymer comprises polycaprolactone (PCL).

24. A method according to any of claims 16 to 23, wherein the solution or suspension comprises elastin, and wherein the elastin is unfractionated and comprises soluble elastin, and optionally wherein the elastin comprises insoluble elastin.

25. A method according to any of claims 16 to 24, wherein the synthetic polymer is, or has been, contacted with the one or more natural polymers, in the absence of any cell.

26. A method according to any of claims 16 to 25, comprising seeding the scaffold, with one or more cells, ex vivo.

27. A method according to claim 26, wherein the one or more cells comprises a stem cell, optionally a human adipose-derived stem cell.

28. A tissue scaffold obtained or obtainable by a method as defined in any of claims 16 to 27.

29. A tissue or cell culture comprising a tissue scaffold as defined in any of claims 1 to 15.

30. A tissue scaffold according to any of claims 1 to 15, for use as a medicament.

31. A tissue scaffold according to any of claims 1 to 15, for use in promoting tissue healing, regeneration or repair.
